(19) United States
(12) Patent Application Publication
(10) Pub. No.: US 2026/0248931 A1
(43) Pub. Date: Aug. 27, 2026

(54) Composition and Method for Synthesizing Pegylated Uricase-Drug Conjugates to Mitigate Anti-Drug Antibody and Their Therapeutic Use

(71) Applicant: OncoC4, Inc., Rockville, MD (US)

(72) Inventors: Yang Liu, Potomac, MD (US); Qiang Yang, Ellicott City, MD (US); Pan Zheng, Potomac, MD (US); Man Bao, Nanjing (CN); Xiao Luo, Nanjing (CN); Qunmin Zhou, Nanjing (CN); Dan Tong, Guangzhou (CN); Xiaoqun Gu, Nanjing (CN)

(21) Appl. No.: 19/532,129

(22) Filed: Feb. 6, 2026

Related U.S. Application Data
(60) Provisional application No. 63/755,875, filed on Feb. 7, 2025.

(51) Int. Cl. A61K 47/64; A61K 9/00; A61K 47/60; A61P 19/06; C12N 9/06

(52) U.S. Cl. CPC A61K 47/64; A61K 9/0019; A61K 47/60; A61P 19/06; C12N 9/0048; C12Y 107/03003

(57) ABSTRACT

The invention relates to uricase-drug conjugates comprising a uricase with a linker-drug attached to either the protein core or other moiety attached to the protein core. The pegylated uricase-drug conjugates mitigate the formation of anti-drug antibodies to improve the therapeutic efficacy of enzymes such as uricase. The uricase-drug conjugate may be administered subcutaneously or intravenously.

### FIELD OF THE INVENTION

The present invention relates to novel pegylated uricase-drug conjugates that mitigate the formation of anti-drug antibodies to improve the therapeutic efficacy of enzymes.

### BACKGROUND OF THE INVENTION

Gout is an afflicting disease characterized by persistent hyperuricemia, caused by the chronic buildup of uric acid crystals in the joints. Symptoms include persistent and severe joint pain, swelling, redness, and sometimes formation of tophi-hard lumps of uric acid crystals around the joints. Among many options for treating gout, recombinant uricase has been successfully applied to the management of hard-to-treat or uncontrolled gout (Schlesinger N et al., Nat Rev Rheumatol. 2023,19:640-649). This enzyme, which can oxidize uric acid to more soluble allantoin, was lost in human and apes during evolution. Pegloticase (Krystexxa®), a chimeric pig/baboon uricase that is pegylated to reduce the immunogenicity and protect the enzyme from cleavage of the serum protease (Sherman M R et al., Adv Drug Deliv Rev. 2008, 60:59-68), was developed by Savient Pharmaceuticals and subsequently approved by FDA for gout in 2008. This pegylated uricase, with a 10 kD linear methoxy PEG (mPEG) attached to the lysine residues of the uricase, forms a homotetramer with a molecular weight of approximately 500 kD (Sherman M R et al., Adv Drug Deliv Rev. 2008, 60:59-68). In mouse studies, pegylation essentially eliminated anti-drug antibodies (ADA) against uricase and increased the serum half-life of enzyme from a few hours to 29 hours (U.S. Pat. No. 6,576,235). Weekly intraperitoneal injections of pegloticase alleviated symptoms in a mouse gout model. Clinically, an 8 mg dose administered every two weeks has proven to be an effective treatment. Despite its clinical success, ADAs are still generated towards pegloticase. Clinical reports indicate that over 40% of patients developed high titers of ADA against pegloticase during treatment and ADA was a significant factor to the non-response and the discontinuation of treatment (Schlesinger N et al., Nat Rev Rheumatol. 2023, 19:640-649). It appears that the majority of ADA is directed against the 10 kD PEG component, not uricase itself (Lipsky P E et al., Arthritis Res Ther. 2014, 16: R60). There is an urgent need to develop a novel chronic treatment for gout that maintains low to zero levels of ADA generation.

### DESCRIPTION OF THE DRAWINGS

FIGS. 1A-C. Design of pegylated uricase-drug conjugates. FIG. 1A) Conjugates with linker-drug directly attached to the lysines of pegylated uricase. FIG. 1B) Conjugates with linker-drug attached to the terminal of 10 kD PEG. FIG. 1C) Choice of DM1, DM4 or other drugs with different linkers.

FIG. 2. Reaction schemes for preparing pegylated uricase-drug conjugates. Reaction Route 1: NHS-mPEG and SMCC-DM1 are simultaneously conjugated to lysine residues of uricase through a succinimidyl carbonate-amine reaction. Reaction Route 2: NHS-mPEG and preassembled NHS-PEG-DM1 were mixed in a 2:1 molar ratio (NHS-mPEG:NHS-PEG-DM1) and conjugated to uricase. Reaction Route 3: 10 kD NHS-mPEG and NHS-PEG-azide were mixed in a 2:1 molar ratio (NHS-mPEG:NHS-PEG-azide) and conjugated to uricase, followed by attachment of BCN-DM1 to terminal of PEG-azide via click chemistry. Reaction Route 4: NHS-PEG and SMCC-DM1 are sequentially conjugated to lysine of uricase.

FIGS. 3A and 3B. Analytical characterization of prepared uricase, pegylated uricase, and pegylated uricase-drug conjugates. FIG. 3A) SDS-PAGE in reducing condition. Lane 1, pegylated uricase-linker-DM1 conjugates produced from reaction route 3 (PEG-uricase-linker-DM1-R3); Lane 2, pegylated uricase-linker-DM1 conjugates produced from reaction route 1 (PEG-uricase-linker-DM1-R1); Lane 3, uricase; Lane 4, pegylated uricase (PEG-uricase). FIG. 3B) Size exclusion chromatography (SEC) analysis.

FIG. 4. Experimental design for in vivo analysis for immunogenicity, pharmacokinetics and pharmacodynamics of uricase-drug conjugates. Uox KO mice of 9-10 weeks old received i.v. injections with either the PEG-Uricase, or PEG-Uricase-linker-DM1-R1 conjugates at 0.3 or 0.4 mg/Kg dose (based on normalized enzymatic activity), Q2W for a total of four injections. Blood samples were collected and analyzed for serum uric acid levels, anti-PEG-Uricase antibody (ADA) concentration and serum PEG-Uricase concentration.

FIG. 5. Immunogenicity of PEG-Uricase and PEG-Uricase-linker-DM1-R1 conjugates. Uox KO mice of 9-10 weeks old received i.v. injections with either with PEG-Uricase, or PEG-Uricase-linker-DM1-R1 conjugates at 0.3 or 0.4 mg/Kg dose (based on normalized enzymatic activity), Q2W for a total of four injections. Serum was diluted 1:40 or 1:50 with PBS containing 0.5% milk, and the serum ADA levels were measured using ELISA method.

FIG. 6. Pharmacokinetics (PK) of PEG-Uricase and PEG-Uricase-DM1-R1 conjugates. Uox KO mice of 9-10 weeks old received i.v. injections with either with PEG-Uricase, or PEG-Uricase-linker-DM1 conjugates at 0.3 or 0.4 mg/Kg dose (based on normalized enzymatic activity), Q2W for a total of four injections. Serum was diluted 1:40 or 1:50 with PBS containing 0.5% milk, and the serum drug levels were measured using ELISA method.

FIGS. 7A and 7B. Pharmacodynamics (PD) of PEG-Uricase and PEG-Uricase-linker-DM1-R1 conjugates. Uox KO mice of 9-10 weeks old received i.v. injections with either with PEG-Uricase, or PEG-Uricase-linker-DM1 conjugates at 0.3 or 0.4 mg/Kg dose (based on normalized enzymatic activity), Q2W for a total of four injections. Serum was diluted 1:10 with double distilled water (ddH₂O), and the serum uric acid levels were measured using the uricase-peroxidase method. Based on the minimum detection limit specified in the uric acid assay kit's instructions, BQL is defined as 60 μmol/L. Data are shown as mean±SD. FIG. 7A). Uric acid concentration per treatment. FIG. 7B). The UA changes from baseline per treatment, which is calculated as the uric acid value before and after each dose divided by the uric acid value before the first dose. Data are shown as mean±SD.

FIGS. 8A, 8B and 8C. Pharmacokinetics (PK) of PEG-Uricase and PEG-Uricase-DM1 Administered Subcutaneously. Uox KO mice of received subcutaneous injections with either PEG-Uricase, or PEG-Uricase-linker-DM1 conjugates, Q1W for a total of six injections (FIG. 8A). Uricase levels during the study period were measured and reported (FIG. 8B). Weight levels during the study period were measured and reported (FIG. 8C).

### DETAILED DESCRIPTION OF THE INVENTION

ADA production starts due to the activation of B cells that express drug-specific antigen receptors on its surface. We recently disclosed these receptors can be used to target these B cells to ablate ADA production, using antigen-drug conjugates comprising antigens covalently linked to cytotoxic drug. To mitigate the generation of ADA against pegloticase, we prepared pegylated uricase-linker-drug conjugates to specifically target pegylated uricase-specific B cells.

The present inventors have now surprisingly found that pegylated uricase-linker-drug conjugates can be used to target B-cells that express drug-specific antigen receptors related to pegylated uricase to ablate the formation of ADA. Provided herein is an uricase-linker-drug conjugate. It should be understood that, in general, the uricase-drug conjugate includes a linker, i.e., uricase-linker-drug, and therefore references to a uricase-drug conjugate should be understood to include a linker unless indicated otherwise. The uricase-linker-drug-conjugate includes uricase, which may comprise an antibody or antigen binding fragment thereof. The uricase may be chemically linked by a linker to one or more drugs or therapeutic agents, which may comprise one or more cytotoxic agents (for example, cytotoxic chemical drugs) and cytostatic agents as a payload. As used herein, payload and drug are used interchangeably. In one example, the uricase drug conjugate comprises uricase, a drug, and a linker that enables attachment or conjugation of the drug to the uricase. The drug should have one or more of: i) sufficiently high cytotoxicity; ii) high stability; and iii) functional groups that can be modified without significantly affecting potency. It should be understood that therapeutic agent, drug, and payload are being used interchangeably herein when referencing the drug component of the uricase-drug conjugate and uricase-linker-drug conjugate.

Other drugs which may be used as the drug in the uricase drug conjugate include one or more of auristatins, auromycins, maytansinoids, topoisomerase I or II inhibitors, ricin, ricin A-chain, combrestatin, duocarmycins, dolastatins, doxorubicin, daunorubicin, taxols, cisplatin, ccl065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At211, Ac225, I131, I125, Y90, Re186, Re188, Sm153, Bi212 or 213, Ra223, Pb212, Tb149, P32 and radioactive isotopes of Lu including Lu177.

The drug may be connected to the uricase via either a cleavable or non-cleavable linker. The linker may form a covalent linkage to the drug at one location and a covalent linkage to the uricase at another location.

The cleavable linker may be hydrazone, disulfide, or peptide linkers. The cleavable linker may further comprise one or more disulfide groups. The non-cleavable linker may be a succinimidyl 4-(N-maleimidomethyl)cyclohexane-1-carboxylate (SMCC) linker. The cleavable linker may also be a peptide linker comprising the dipeptide linker, Val-Cit-PAB ((2S)-5-((aminocarbonyl)amino)-2-(((2S)-2-amino-3-methylbutanoyl)amino)-N-(4-(hydroxymethyl)phenyl) pentanamide) and Val-Ala-PAB ((S)-2-Amino-N—((S)-1-((4-(hydroxymethyl)phenyl)amino)-1-oxopropan-2-yl)-3-methylbutanamide). The cleavable linker may be a disulfide linker comprising reducible or glutathione-sensitive disulfide linkers. In one aspect, the reducible or glutathione-sensitive disulfide linker is SPDB (butanoic acid, 4-(2-pyridinyldithio)-, 2,5-dioxo-1-pyrrolidinyl ester). The cleavable linker may be CL2A ((6,12,15,18,21,24,27,30,33-Nonaoxa-3,9-diazapentatriacontanamide, 2-(4-aminobutyl)-35-[4-[[[[4-[(2,5-dihydro-2,5-dioxo-1H-pyrrol-1-yl)methyl]cyclohexyl] carbonyl]amino]methyl]-1H-1,2,3-triazol-1-yl]-N-[4-(hydroxymethyl)phenyl]-4,8-dioxo-, (2S)-). The cleavable linker may be mc-GGFG ((S)-6-(2,5-Dioxo-2,5-dihydro-1H-pyrrol-1-yl)-N-(2-((2-((1-((2-((4-(hydroxymethyl)phenyl)amino)-2-oxoethyl)amino)-1-oxo-3-phenylpropan-2-yl)amino)-2-oxoethyl)amino)-2-oxoethyl) hexanamide).

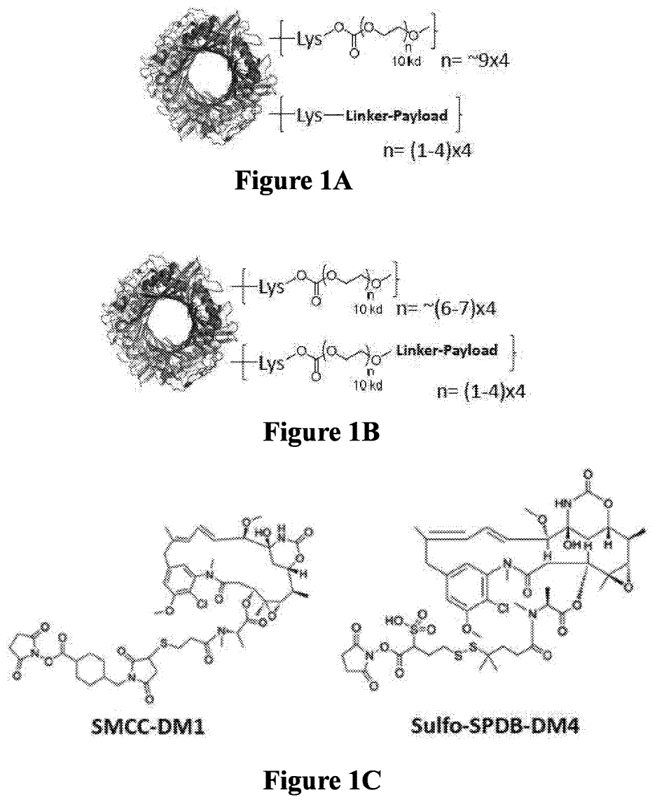

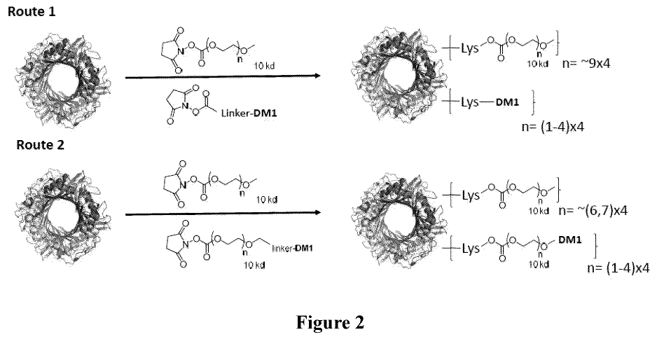

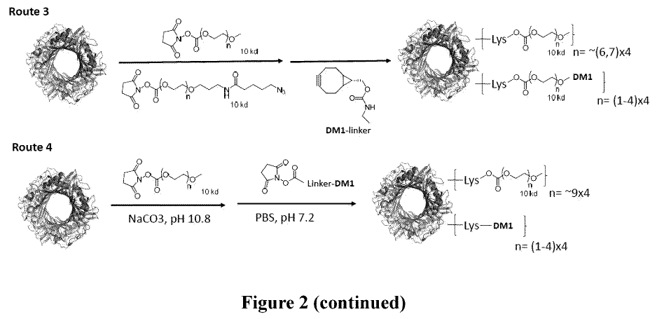

To generate pegylated uricase-linker-drug conjugates, we first designed four different synthesis schemes to conjugate DM1 with a non-cleavable linker or DM4 with a cleavable linker to pegylated uricase to form pegylated enzyme-drug conjugates (FIGS. 1A-C & FIG. 2). By design, the drug or drug-linker are attached to either the lysine residues of uricase or to the terminal of 10 kD PEG chain (FIGS. 1A and 1B). Simple general structures of two of the possible pegylated uricase-drug conjugates are shown below as Compounds A and B. It should be understood that only single PEG conjugates and drug-linker conjugates are illustrated but multiple of each will be present on the various monomers of uricase.

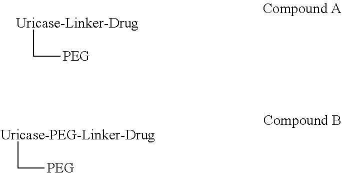

FIG. 1A illustrates the 10 kD PEG chain covalently attached to a lysine residue of uricase and the linker and drug covalently attached to a different lysine residue of uricase. Uricase is a homotetramer protein of four identical 34 kD subunits. Although FIG. 1A illustrates a single PEG chain and a single linker-drug, it should be understood that multiple PEG chains and linker-drugs are attached to lysine residues of each homotetramer uricase. In FIG. 1A, the notation n=~9×4 for the PEG chain indicates that pegylated uricase includes about nine PEG chains on each of the four homotetramers. FIG. 1A also includes 1 to 4 drug conjugates (i.e., linker and drug) on each of the four homotetramers. Similarly, FIG. 1B illustrates that the uricase has 6-7 PEG chains on each homotetramer and a drug conjugate (i.e., linker and drug) attached to 1-4 different PEG chains on each of the four homotetramers. This same terminology is used in FIG. 2.

For the choice of linker-drug, SMCC-DM1 with non-cleavable linker or SPDB-DM4 with a reducible linker (FIG. 1C) are used. We designed four conjugation routes, as shown in (FIG. 2). In Route 1, the linker and drug (i.e., cytotoxin) was co-conjugated to lysine residues of the uricase together with 10 kD methoxy PEG (mPEG), via the same succinimidyl carbonate-amine conjugation chemistry. In Route 2, the 10 kD NHS-mPEG and NHS-PEG-linker-DM1 (with the payload preassembled to the terminal of NHS-PEG) were mixed in the molar ratio of 2:1 (NHS-mPEG: NHS-PEG-linker-DM1) and then coupled to the uricase, resulting in the cytotoxin being attached to the end of some of the PEG chains. In Route 3, the 10 kD NHS-mPEG and NHS-PEG-azide were mixed in a molar ratio of 2:1 (NHS-mPEG: NHS-PEG-azide) and then coupled to the uricase. The azido pegylated-uricase was then clicked with BCN-DM1 via strain-promoted azide alkyne cycloaddition (SPAAC) (“click chemistry”), similarly attaching the cytotoxin to the end of the PEG chain. This synthetic route retains the flexibility for changing drugs (e.g., payloads) or if the synthesis of NHS-PEG-linker-drug is challenging. In any route, the drug-enzyme molar ratio was controlled between 1-4 drug per monomer of uricase. During experimentation, we unexpectedly observed that 10 kD NHS-mPEG and small SMCC-DM1 have different site preferences for lysine sites. Therefore, we developed route 4, in which NHS-mPEG was attached first, followed by the addition of SMCC-DM1.

### Production of Uricase

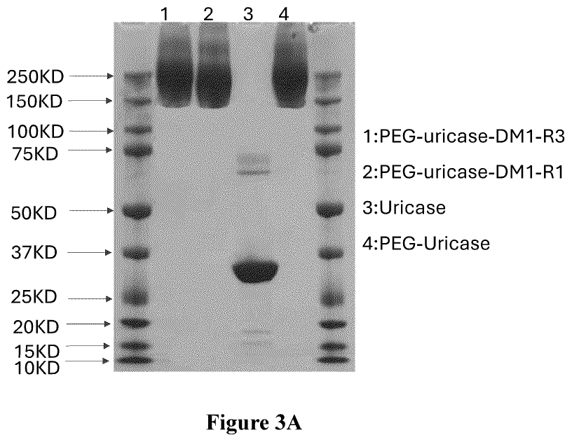

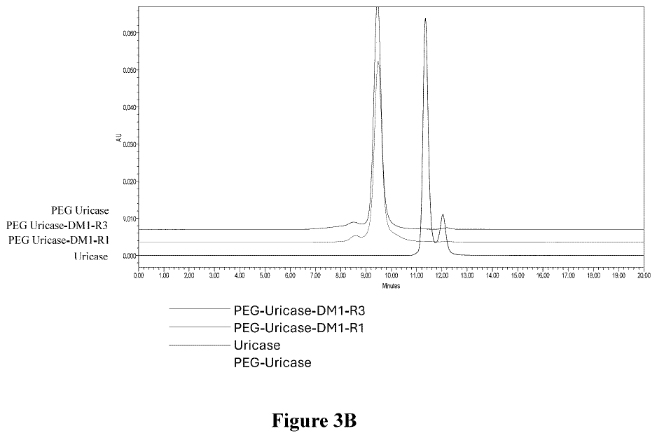

The chimeric pig/baboon uricase was produced by recombinant expression in E. coli. Briefly, the chimeric uricase was expressed as an insoluble protein in the inclusion body. After lysing of E. coli cells, the inclusion body, mainly composed of the uricase, was dissolved in a sodium carbonate buffer, pH 10.3. The dissolved crude uricase was purified with preparative ion exchange chromatography. Fractions containing uricase activity were collected and exchanged to sodium carbonate buffer, pH 10.3. The enzyme activity was analyzed by a UV assay to measure the increase of the product allantoin at 292 nm (Fridovich I. J Biol Chem. 1965, 240:2491-4). The size and aggregation level were determined by SDS-PAGE and size exclusion chromatography (SEC) (FIG. 3A).

### Example 1

### Preparation of the Pegylated Uricase-Linker-Drug Conjugates

In the reaction Route 1, NHS-mPEG and SMCC-DM1 were dissolved in DMSO and added to a uricase solution in a final molar ratio of 24:3:1 (NHS-mPEG: SMCC-DM1: uricase). The reaction mixture was incubated at room temperature (RT) for 60 min with end-to-end rotation. The conjugation was stopped by quenching with 0.1 M Tris-HCl. The unreacted 10 kD NHS-mPEG and SMCC-DM1 were removed by SEC. The conjugate was exchanged to a buffer of 20 mM NaPO4, 150 mM NaCl, pH 7.2 by three rounds of centrifugation/dilution with a 30 kD centrifugal filter. The uricase activity of enzyme-drug conjugates, by UV (292 nm) measurement of the oxidation of uric acid to allantoin, was generally between 3-6 U/mg. The pegylation was assessed by SDS-PAGE (FIG. 3A). It is estimated that each uricase monomer carries more than 8 strands of 10 kD PEG. The pegylated uricase-linker-DM1 conjugate appeared to be a single homotetramer, with less than 5% of aggregates, as illustrated in the SEC analysis (FIG. 3B). The drug-enzyme ratio (DER) was determined by a UV assay as in a range of 1.3-1.5.

As shown in Table 1, in comparison with the pegylated uricase, conjugation to the cytotoxic drug caused only a minimal reduction of enzymatic activities.

For reaction Route 2, NHS-mPEG and preassembled NHS-PEG-linker-DM1 were mixed and added to uricase solution in a final molar ratio of approximately 18:9:1 (NHS-mPEG: NHS-PEG-linker-DM1: uricase). The ratio may be optimized to achieve an optimal DER based on minimal loss of enzymatic activities and maximal inhibition of ADA.

In reaction Route 3, NHS-mPEG and NHS-PEG-azide powder were mixed and added to uricase solution in a final molar ratio of 18:9:1 (NHS-mPEG: NHS-PEG-azide: uricase). After pegylation, BCN-DM1 was added to azido-uricase solution in a molar ratio of 25:1 (BCN-DM1: uricase) and then was clicked with BCN-DM1 by reaction at room temperature (RT) for overnight. The ratio may be optimized to achieve an optimal DER based on minimal loss of enzymatic activities and maximal inhibition of ADA.

In reaction Route 4, NHS-mPEG powder was added into a uricase solution in a final molar ratio of 27:1 (NHS-mPEG: uricase) and reacted in the same condition as that of Route 1. After SEC removal of unreacted PEG and buffer-exchange to sodium phosphate buffer pH 7.2 or sodium carbonate buffer pH 10.8, the uricase-PEG was reacted with SMCC-DM1 dissolved in DMSO at molar ratios of 1:3, 1:6, 1:9 and 1:12 (uricase-PEG: SMCC-DM1). After removal of unreacted SMCC-DM1 with a centrifugal filter, the DER was determined to be in a range of 1.38-6.26. In the conjugation reaction at pH 7.2 using phosphate buffer (PB), the enzyme activity did not decrease with the increase of DER, as shown in Table 2. In contrast, while the use of a carbonate buffer at pH 10.8 yielded a higher DER overall, there appears to be an inverse correlation between DER and enzymatic activity.

To prepare a reference pegylated uricase (as a surrogate for pegloticase), NHS-mPEG powder was added into uricase solution in a final molar ratio of 27:1 (NHS-mPEG: uricase) and reacted in the same condition as that of Route 1. After SEC removal of unreacted PEG, the pegylated uricase was exchanged to a buffer of 20 mM NaPO4, 150 mM NaCl, PH 7.2.

### Example 2

### Evaluation of Immunogenicity, PK and PD in a Mouse Gout Model

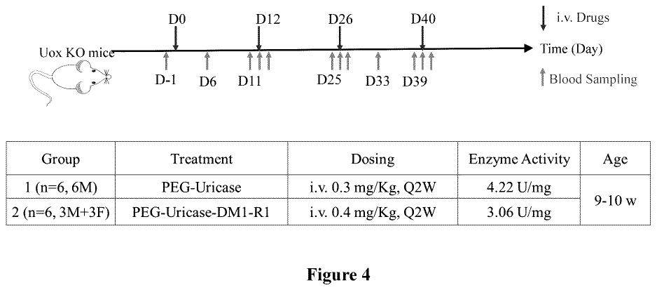

A uricase deficient gout mouse model (“Uox KO”) was used to evaluate the immunogenicity, pharmacokinetics (PK) and pharmacodynamics (PD) of pegylated uricase of pegylated uricase-linker-DM1 conjugates generated via Route 1. In this mouse model, the homozygous deletion of mouse uricase gene results in persistent hyperuricemia. Briefly, Uox KO mice of 9-10 weeks old received i.v. injections with either with PEG-Uricase, or PEG-Uricase-linker-DM1-R1 conjugates at 0.3 or 0.4 mg/Kg dose (based on normalized enzymatic activity), once every two weeks (Q2W) for a total of four injections. The blood samples were collected for ADA, PK and PD, as diagramed in FIG. 4.

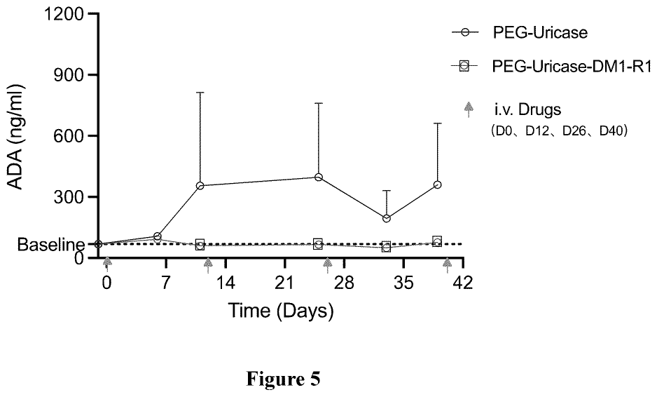

To compare immunogenicity, sera were diluted 1:40 or 1:50 with PBS containing 0.5% milk and the serum ADA levels were measured using ELISA method and the antibody concentration was determined using reference standard. As shown in FIG. 5, while significant and sustained ADA was induced by the pegylated uricase starting as early as Day 11 after drug treatment, the PEG-Uricase-linker-DM1-R1 induced no ADA throughout the study period. These data demonstrate effective ablation of ADA through the use of a PEG-Uricase-linker-DM1 conjugate.

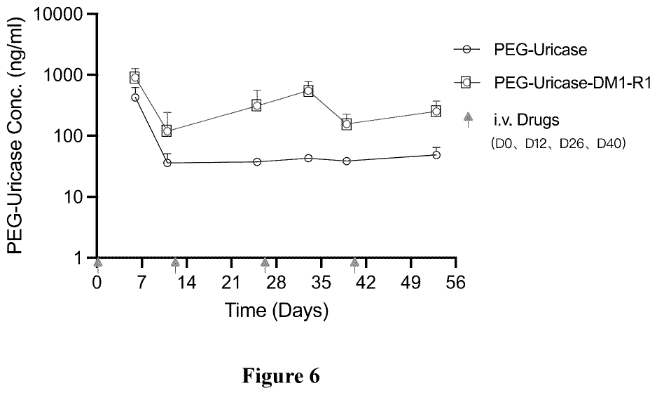

To determine the impact of ADA on drug PK, mouse sera were diluted 1:20 with PBS containing 0.5% milk, and the serum PK levels were measured using the ELISA method. As shown in FIG. 6, while sustained drug accumulation was achieved in the pegylated uricase-linker-DM1-R1-treated mice, no drug accumulation was achieved in the pegylated uricase-treated mice. These data confirmed that ADA induction by the pegylated uricase caused clearance of the drug. Conversely, due to the ablation of ADA as a result of the administration of the pegylated uricase-linker-DM1-R1 a sustained accumulation of pegylated uricase-linker-DM1 was seen.

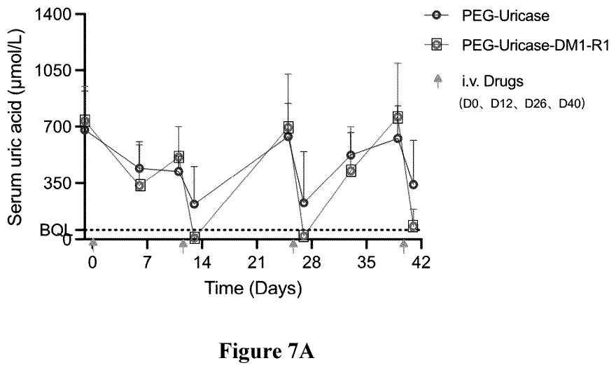

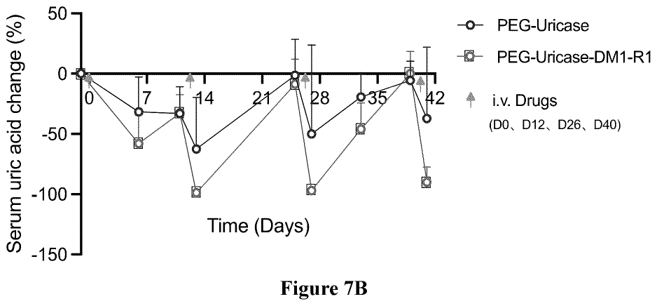

Consistent with the sustained increase in drug accumulation seen due to an ablated ADA response, pegylated uricase-linker-DM1-R1 shows increased therapeutic activity versus pegylated uricase. As shown in FIGS. 7A and 7B, starting from second injection cycle, pegylated uricase-linker-DM1-R1 reduced uric acid to undetectable levels at 24 hours post second treatment; whereby an equal amount of pegylated uricase failed to reduce uric acid by more than 50% levels at any time throughout the first four cycles.

Taken together these data demonstrate that pegylated uricase-linker-DM1-R1 is largely devoid of immunogenicity and conferred stronger therapeutic activity versus standard pegylated uricase. In contrast, pegylated uricase without a cytotoxic drug payload conjugated to it shows strong immunogenicity, as observed also in humans, and thus has significantly less potent and/or stable therapeutic activity.

### Example 3

### Evaluation of Subcutaneous Administration in a Mouse Gout Model

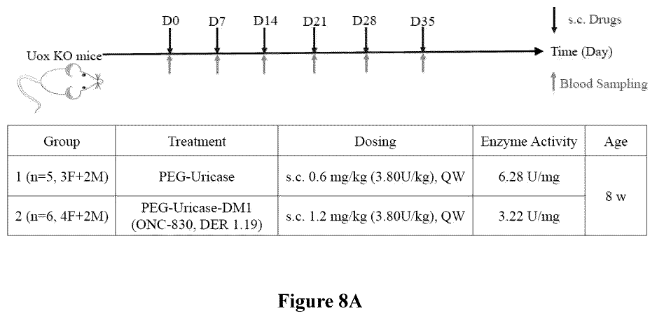

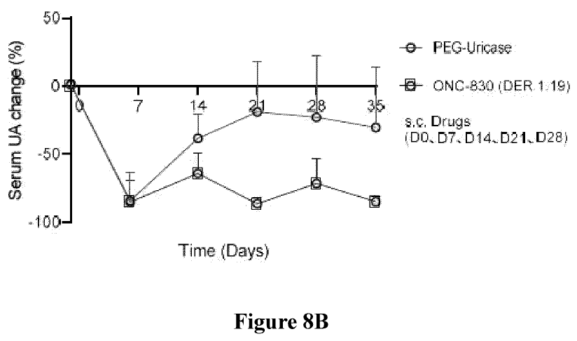

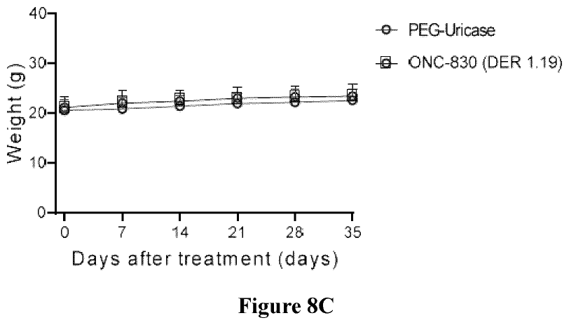

In this second mice study, we further compared the pharmacodynamic (PD) activity and tolerability (body weight change) of PEG-uricase and PEG-uricase-DM1 in Uox knockout (KO) mice. To align with the intended clinical route of administration, Uox KO mice (~8 weeks of age) were dosed subcutaneously (SC). To explore a more durable and complete urate-lowering effect, the dosing route was changed from IV to SC with once-weekly (QW) administration, and the dose was increased to 3.80 U/kg. The experimental design and dosing schedule are shown in FIG. 8A. As illustrated in FIG. 8A, the mice were administered the treatment at Days 0, 7, 14, 21, 28 and 35. Blood samples were taken on each of those days as well. The control group of mice received PEG-Uricase at 0.6 mg/kg (3.80 U/kg) once weekly with an enzyme activity of 6.28 U/mg. The test group of mice received PEG-Uricase-DM1 (ONC-830, DER 1.19) once weekly with an enzyme activity of 3.22 U/mg.

Over the observation period through Day 35, PEG-uricase-DM1 demonstrated a more pronounced and sustained urate-lowering effect compared with PEG-uricase. See FIG. 8B. While both treatments initially reduced serum uric acid (sUA) levels, the PEG-uricase group began to lose efficacy around Day 14, with sUA levels rebounding toward baseline by Day 35, likely due to the development of neutralizing antibodies and/or accelerated clearance. In contrast, the PEG-uricase-DM1 group maintained a near-complete reduction in SUA level throughout the 35-day study period. These data suggest that subcutaneous administration of PEG-uricase-DM1 reduces the ADA and provides efficacy over an extended period.

In the same study, as illustrated in FIG. 8C, body weight measurements showed no apparent treatment-related body weight loss in the PEG-uricase-DM1 group compared with the PEG-uricase group over the dosing period. These data suggest that the DM1-conjugated enzyme was well tolerated at the therapeutic dose.

Taken together, the results of the studies reported in Examples 2 and 3 demonstrate that PEG-Uricase-DM1 can be administered intravenously and that treatment effect extends equally well to subcutaneous injection. Thus, a patient and physician has the option of either intravenous injection or the more convenient subcutaneous injection.

Based on the above studies, the inventors believe the compositions disclosed herein, Uricase-drug conjugates comprising a uricase with a linker-drug attached to either the protein core or other moiety attached to the protein core, can be administered to humans both intravenously and subcutaneously to mitigate ADA levels.

>pig/baboon uricase
SEQ ID NO: 1
TYKKNDEVEFVRTGYGKDMIKVLHIQRDGKYHSIKEVATTVQLTLS
SKKDYLHGDNSDVIPTDTIKNTVNVLAKFKGIKSIETFAVTICEH
FLSSFKHVIRAQVYVEEVPWKRFEKNGVKHVHAFIYTPTGTHFCE
VEQIRNGPPVIHSGIKDLKVLKTTQSGFEGFIKDQFTTLPEVKDR
CFATQVYCKWRYHQGRDVDFEATWDTVRSIVLQKFAGPYDKGEYS
PSVQKTLYDIQVLTLGQVPEIEDMEISLPNIHYLNIDMSKMGLIN
KEEVLLPLDNPYGKITGTVKRKLSSRL

### DEFINITIONS OF TERMS

PEG—Polyethylene glycol
ADA—Anti-drug antibodies
NHS—N-hydroxysuccinimide
mPEG—methoxy PEG
NHS—mPEG-methoxy PEG NHS ester
NHS—PEG-DM1-PEG NHS ester preassembled with DM1
SMCC—succinimidyl 4-(N-maleimidomethyl)cyclohexane-1-carboxylate
SPDB—butanoic acid, 4-(2-pyridinyldithio)-, 2,5-dioxo-1-pyrrolidinyl ester
BCN—bicyclo[6.1.0]non-4-yne
kD—kilodaltons
SDS-PAGE—sodium dodecyl sulfate-polyacrylamide gel electrophoresis
SEC—Size Exclusion Chromatography
R1—Reaction Route 1
R3—Reaction Route 3
PBS—Phosphate buffered saline
ONC-830—PEG-Uricase-linker-DM1
ddH2O—double distilled water
cc I065—Rachelmycin
PE40—a modified Pseudomonas exotoxin
DMSO—dimethyl sulfoxide

We claim:

1. Uricase-drug conjugates comprising a uricase with a linker-drug attached to either the protein core or other moiety attached to the protein core.

2. The uricase-drug conjugates of claim 1, wherein the uricase comprises a chimeric pig/baboon enzyme having an amino acid sequence set forth in SEQ ID NO: 1.

3. The uricase-drug conjugates of claim 1, wherein the drug is pegylated.

4. The uricase-drug conjugated in claim 3, where the drug is pegylated with PEG comprising 5-10 kD linear PEG.

5. The pegylated uricase-drug conjugates of claim 4, where each uricase monomer is attached with at least six strands of PEG.

6. The pegylated uricase-drug conjugates of claim 1, wherein the drug is toxic to a B-cell that produces anti-drug antibodies to the pegylated uricase.

7. The pegylated uricase-drug conjugates of claim 6, wherein the drug comprises auristatins, auromycins, maytansinoids, topoisomerase I or II inhibitors, ricin, ricin A-chain, combrestatin, duocarmycins, dolastatins, doxorubicin, daunorubicin, taxols, cisplatin, ccl065, ethidium bromide, mitomycin, etoposide, tenoposide, vincristine, vinblastine, colchicine, dihydroxy anthracin dione, actinomycin, diphtheria toxin, Pseudomonas exotoxin (PE) A, PE40, abrin, abrin A chain, modeccin A chain, alpha-sarcin, gelonin, mitogellin, retstrictocin, phenomycin, enomycin, curicin, crotin, calicheamicin, Sapaonaria officinalis inhibitor, and glucocorticoid and other chemotherapeutic agents, as well as radioisotopes such as At211, Ac225, I131, I125, Y90, Re186, Re188, Sm153, Bi212 or 213, Ra223, Pb212, Tb149, P32 and radioactive isotopes of Lu including Lu177.

8. The drug of claim 7, wherein the drug comprises mytansoids DM1 or DM4.

9. The drug of claim 7, wherein the drug comprises a topoisomerase I inhibitor.

10. The drug of claim 9, wherein the topoisomerase I inhibitor comprises SN-38, belotecan, Dxd, or exatecan.

11. The uricase-drug conjugate of claim 1, wherein the drug is conjugated to uricase via a cleavable or non-cleavable linker.

12. The linker of claim 11, wherein the linker forms a covalent linkage to the drug at one location and a covalent linkage to the uricase at another location.

13. The linker of claim 11, wherein the linker is non-cleavable.

14. The linker of claim 13, wherein the non-cleavable linker comprises a succinimidyl 4-(N-maleimidomethyl)cyclohexane-1-carboxylate (SMCC) linker.

15. A process of making the uricase-drug conjugate of claim 1, the process comprising preparation by simultaneous conjugation of both the linker-drug and the PEG to one or more lysine residues of the uricase.

16. A process of making the uricase-drug conjugate of claim 1, the process comprising simultaneous conjugation of the PEG and PEG pre-conjugated with the linker-drug in a suitable molar ratio to the uricase, the process resulting in the linker-drug being attached to the terminal end of the PEG chain.

17. A process of making the uricase-drug conjugate of claim 1, the process comprising simultaneous conjugation of the PEG and activated PEG in a suitable molar ratio to the uricase, followed by coupling of the linker-drug to the activated PEG, resulting in the linker-drug being attached to the terminal end of the PEG chain.

18. A process of making the uricase-drug conjugate of claim 1, the process comprising preparing a sequential conjugation of both linker-drug and the PEG to the lysine residues of the uricase.

19. A method of treating gout patient with administration of the uricase-drug conjugate in claim 1 at a clinically effective dose and frequency.

20. The method of claim 19, wherein the uricase-drug conjugate is administered subcutaneously or intravenously.
